SEQUENCE LISTING (2 sequences)

SEQ ID NO: 1 - DNA, 25 bp, synthetic construct
  source 1..25: mol_type = other DNA
cagtcatatgaatctgctggatccg

SEQ ID NO: 2 - DNA, 28 bp, synthetic construct
  source 1..28: mol_type = other DNA
catactcgagtcatttatagtccggatg

(19) United States
(12) Patent Application Publication
(10) Pub. No.: US 2024/0141299 A1
(43) Pub. Date: May 2, 2024

(54) Cofactors FAD and PLP for Promoting Pancreatic Progenitors in Pancreatic Development

(71) Applicant: Qatar Foundation for Education, Science and Community Development, Doha (QA)

(72) Inventors: Prasanna R. Kolatkar, Doha (QA); Essam M. Abdelalim, Doha (QA); Noura Aldous, Doha (QA); Zeyaul Islam, Doha (QA)

(21) Appl. No.: 18/498,404

(22) Filed: Oct. 31, 2023

Related U.S. Application Data
(60) Provisional application No. 63/421,394, filed on Nov. 1, 2022.

(51) Int. Cl. C12N 5/071; A61K 35/39

(52) U.S. Cl. CPC C12N 5/0676; A61K 35/39; C12N 2500/38; C12N 2500/40; C12N 2506/45

(57) ABSTRACT

The instant disclosure relates to methods and compositions for increasing β-cell production from stem cells.

### CROSS REFERENCE TO RELATED APPLICATIONS

This application claims the benefit of U.S. Provisional Application No. 63/421,394 filed Nov. 1, 2022, which is incorporated herein by reference in its entirety.

### SEQUENCE LISTING

A Sequence Listing is submitted herewith and incorporated by reference herein as an XML file created on Oct. 30, 2023, entitled “5600234-01072_Sequence_Listing.xml” and having a size of 3 KB.

### BACKGROUND

Induced pluripotent stem cell (iPSC) development is a key process being studied widely due to the potential for modulating these pathways towards specific differentiation outcomes such as cardiac or pancreatic tissues for regenerative medicine. Although a multitude of proteins play major roles in these processes, a critical role is played by transcription factors. Transcription factors (TFs) comprise a large percentage of the total genes and their interplay during development is an important step towards differentiation.

Among the TFs the family of SOX proteins plays significant roles in diverse pluripotency and differentiation programs despite having highly conserved sequences within this family. Several studies have shown how single residue changes within the TF or single base changes in the respective genomic binding site can subtly alter the mutual recognition and result in completely changed differentiation outcomes. SOX2 and SOX17 can adopt each other's programs through single site changes within the HMG domain (DNA-binding) or the cognate cis element.

Combinatorial TF interactions drive the majority of transcriptional pathways at promoters and also at distal enhancer regions. Little is known of potential small-molecule metabolic factors which could potentially affect TF pathways. Nuclear receptor HNF4a has been shown to be modulated by endogenous linoleic acid using mass spectrometry and could play a role in hepatic lipid metabolism). MocR-like bacterial transcription factors (MocR-TFs) comprise N-terminal DNA-binding domains and a pyridoxal 5′-phosphate (PLP) binding site at the C-terminal portion. The capability to recognize specific DNA sequences and concurrently bind PLP allow these TFs to regulate transcription of different metabolic processes involving vitamin B6 and amino acids.

ID2 was also reported to be functionalized through a divalent ion. However, these are unique and rare endogenous small-molecule metabolic factors which are known to interact with a TF and modulate the TF pathway.

SOX9, a member of the SOXE family, is known to be important in pancreatic development. A key role of SOX9 is to maintain pancreatic progenitor cells but also has additional roles in endocrine differentiation. SOX9 also plays a role in hepatic and duodenal programs.

Dimerization of SOX9 was observed to be important for its biological function and A76E mutation was shown to disrupt dimerization and its function. Dimerization could also be installed via interaction of HMG domain and the dimerization domain of SOX9. SOX9 is shown to predominantly bind at promoters during foregut development.

β-cell neogenesis has been applied to create in-vitro methods for producing glucose responsive β-cells. Adult pancreas also exhibit β-cell neogenesis under certain conditions such as partial duct ligation or cerulein treatment. There is a need for better understanding of mechanisms involved in β-cell production in order to improve treatment outcomes either through better in vitro production of β-cells or in situ production of β-cells within the adult pancreas.

### SUMMARY

The present disclosure generally relates to the use of cofactors to increase efficiency in producing β-cells from stem cells including human induced progenitor stem cells and human embryonic cells. Disclosed embodiments comprise compositions and methods of use. For example, disclosed compositions can comprise cofactors such as Flavin Adenine Dinucleotide (FAD) and Pyridoxal 5′-phosphate (PLP).

Disclosed methods of use can comprise the use of cofactors such as FAD and PLP to increase efficiency in producing β-cells.

Disclosed embodiments comprise kits, for example kits comprising cofactors such as FAD and PLP for use in increasing efficiency in producing β-cells.

### DESCRIPTION OF DRAWINGS

FIG. 1A and FIG. 1D show SOX9 HMG domain produced using bacterial expression. FIG. 1B shows vials for absorbance spectroscopy. FIG. 1C and FIG. 1F show SOX2 HMG domain produced using bacterial expression. FIG. 1E shows absorbance at 420 nm.

FIG. 2 shows Mass spectrometry of SOX9-HMG-sox and the table shows the FAD binding site.

FIG. 3 shows SOX9 mutant data showing a loss of absorbance when the FAD binding residue is mutated.

FIG. 4 shows gene expression of differentiated cells during early stages of pancreatic differentiation (days 8, 10 and 12). High expression of pancreatic and duodenal homeobox 1 (PDX1), SOX9 and Homeobox protein NKX6.1 (NKX6.1) is observed at day 12 of differentiation. GAPDH is used as housekeeping gene.

FIG. 5 shows relative changes in neurogenin-3 (NGN3) and NKX6.1. Addition of FAD to early stage differentiation appears to increase pancreatic developmental factors.

FIG. 6A shows a schematic for treatment of hiPSCs with cofactors. FIG. 6B shows the efficiency of pancreatic progenitor differentiation as determined by examining the expression of PDX1 and NKX6.1.

FIG. 7 shows western blotting experiments demonstrating increased levels of the transcription factors, SOX9 and PTF1a, upon FAD treatment at 0.5 μM and 1 μM concentrations.

FIG. 8A shows expression of PTF1a at about 0.5 μM-1 μM concentration of FAD. FIG. 8B shows expression of PTF1a at about 4 μM-8 μM concentration of PLP.

### DETAILED DESCRIPTION

The pancreas has two main functions: an exocrine function that helps in digestion and an endocrine function that regulates blood glucose. β-cells are endocrine cells in the pancreas that produce and release insulin in response to blood glucose levels. In people with type 2 diabetes, β-cells have to work harder to produce enough insulin to control high blood sugar levels. This can lead to reduced β-cell mass through apoptosis, necrosis, and/or autophagy. In people with type 1 diabetes, the pancreas makes little or no insulin. Transplantation of ex-vivo cultured β-cells provides an opportunity for curing type 1 diabetes.

Disclosed herein is the use of cofactors, for example such as FAD and PLP, to increase the efficiency in producing β-cells from stem cell cultures, for example by inducing the production of cells expressing a large increase in certain markers of pancreatic development. Pancreatic cells may be endocrine cells (or insulin producing cells or β-cells) or pancreatic cells may be exocrine cells which do not play a role in insulin production.

In human pancreatic islet, NKX6.1 expression is exclusive to β cells and is undetectable in other islet cells. Activation of NKX6.1 in pancreatic progenitors expressing PDX1 (PDX1+/NKX6.1+) is indicative of future commitment to monohormonal β cells. Provided are methods that allow for selectively producing the endocrine (insulin producing) β-cells. It has been found that expression of neurogenin-3 (NGN3) is predictive of endocrine lineage in differentiating iHPSCs. Further, the fold-expression of NGN3 is higher than NKX6.1, so NGN3 may be a more sensitive biomarker for identifying pancreatic differentiation.

In one embodiment, described herein is the role of the cofactor FAD which binds to SOX9 and modulates the subsequent binding to its cis element, thereby affecting the function at the cellular level. During purification of SOX9 HMG domain from bacterial expression, a yellowish-brown color was seen in the purified protein. By contrast, purified SOX2 HMG protein was clear, as also seen previously with other SOX HMG domains such as SOX17 and SOX4. Absorbance spectroscopy of SOX9 HMG domain also showed a peak at ˜420 nm corresponding to potential FAD cofactor while the SOX2 HMG domain showed no corresponding peak at ˜420 nm.

MALDI-TOF was subsequently applied to further confirm the putative bound cofactor. The mass spectroscopic analysis showed that there were 6 significant peaks corresponding to FAD, Flavin Mononucleotide (FMN) and PLP binding. Mutant proteins were made at the putative site of cofactor binding for all 6 peptides identified by 1D-PAGE in-gel digest and LC-MS/MS (K122, K141, K166, K167, H104, H165). The 6 mutant proteins as well as a dimerization mutant (A76E) were subjected to absorbance spectroscopy. Dimerization of SOX9 is known to play a role in chondrogenesis. The characteristic peak expected at ˜420 nm for the presence of any cofactors was lost only in the mutants associated with binding of FAD (H104, H165) as well as in the dimerization mutant.

Sequence analysis showed that SOX9 residues H104 and K166 as well as A76 were the rare residues not conserved when compared to all the other SOX family members. This is a highly conserved family with >90% identity in the HMG domain. Available structures for SOX9 HMG domain also showed that unaccounted density was present near the H104, H165 residues which could potentially be FAD bound to SOX9 HMG domain. In addition these non-conserved residues could explain the reason that FAD binds specifically to the SOXE family and no other SOX members.

Accordingly described herein are methods for contacting human induced pluripotent stem cells (hiPSCs) with FAD at an early pancreatic progenitor stage, thereby increasing the pancreatic progenitor population, increasing differentiation efficiency, promoting development by increasing expression of SOX9, and subsequently increasing β-cell regeneration.

Decreased PLP plays a role in diabetes due to potential alterations in enzymes related to disturbed insulin activity. Accordingly described herein are methods for contacting hiPSCs with PLP at an early pancreatic progenitor stage, thereby increasing the pancreatic progenitor population, increasing differentiation efficiency, promoting development by increasing expression of SOX9, and subsequently increasing β-cell regeneration.

### Definitions

As used herein “pancreatic progenitor stage of differentiation” refers to the stage S4 typically observed during differentiation of ihPSCs. There are five distinct sequential differentiation stages: first, the conversion of undifferentiated hESCs into definitive endoderm (S1), followed by primitive gut tube endoderm (S2), posterior foregut endoderm (S3), pancreatic progenitors (S4), and finally hormone-positive cells (S5).

“Endocrine pancreatic β-cells” refers to cells that can produce insulin.

The term “therapeutically effective amount” means an amount which, when administered to the subject, results in beneficial or desired results, including clinical results, e.g., inhibits, suppresses or reduces the symptoms of the condition (e.g., diabetes, pancreatic cancer) being treated in the subject as compared to a control. For example, a therapeutically effective amount can be given in unit dosage form (e.g., 0.1 mg to about 50 g per day, alternatively from 1 mg to about 5 grams per day; and in another alternatively from 10 mg to 1 gram per day). The therapeutically effective amount administered to the subject will depend on the mode of administration, the type, and severity of the disease, and on the characteristics of the subject, such as general health, age, sex, body weight, and tolerance to drugs. The skilled artisan will be able to determine appropriate dosages depending on these and other factors. “Therapeutic composition” refers to a composition comprising a therapeutically effective amount of endocrine pancreatic β-cells.

“Treating” or “treatment” refers to obtaining a desired pharmacological and/or physiological effect. The effect can be therapeutic, which includes achieving, partially or substantially, one or more of the following results: partially or substantially reducing the extent of the disease; ameliorating or improving a clinical symptom or indicator associated with the disease; delaying, inhibiting or decreasing the likelihood of the progression of the disease; or decreasing the likelihood of recurrence of the disease.

The terms “administer”, “administering”, “administration”, and the like, as used herein, refer to methods that may be used to enable delivery of compositions to the desired site of biological action. The particular mode of administration and the dosage regimen will be selected by the attending clinician, taking into account the particulars of the case.

“Pharmaceutically acceptable carrier” refers to a substance that aids the formulation and/or administration of a cofactor to and/or absorption by a subject and can be included in the pharmaceutical compositions of the disclosure without causing a significant adverse toxicological effect on the subject. Non limiting examples of pharmaceutically acceptable carriers and/or diluents include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, hydroxymethycellulose, fatty acid esters, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with or interfere with the activity of the compounds provided herein. One of ordinary skill in the art will recognize that other pharmaceutical excipients are suitable for use with disclosed compounds or pharmaceutically acceptable salts thereof.

### Methods

Provided is a method for increasing efficiency in producing β-cells, comprising treating pancreatic progenitor cells with at least one cofactor.

Also provided is a method for producing endocrine pancreatic β-cells, the method comprising
contacting a culture including human induced pluripotent stem cells (hiPSCs) with at least one cofactor;
differentiating at least a portion of the hiPSCs to endocrine pancreatic 3-cells; and
measuring expression of a biomarker selected from PDX1, NKX6, NGN3, or a combination thereof to identify the endocrine pancreatic β-cells, wherein expression of said biomarkers indicates differentiation to endocrine pancreatic β-cells.

In some embodiments, the method comprises differentiating at least a portion of the hiPSCs to endocrine pancreatic β-cells; and measuring expression of PDX1. In some embodiments, the method comprises differentiating at least a portion of the hiPSCs to endocrine pancreatic β-cells; and measuring expression of NKX6 (including NKX6.1). In some embodiments, the method comprises differentiating at least a portion of the hiPSCs to endocrine pancreatic β-cells; and measuring expression of NGN3. In some embodiments, the method comprises differentiating at least a portion of the hiPSCs to endocrine pancreatic β-cells; and measuring expression of PTF1a wherein identification of PTF1a indicates potential loss of endocrine lineage. In some embodiments, the method comprises differentiating at least a portion of the hiPSCs to endocrine pancreatic β-cells; and measuring expression of SOX9 wherein identification of PTF1a indicates potential loss of endocrine lineage.

In some embodiments, the method is a method for identifying and/or predicting a commitment to differentiation of hiPSCs into endocrine pancreatic β-cells.

In some embodiments, the at least one cofactor is Flavin Adenine Dinucleotide (FAD), Pyridoxal 5′-phosphate (PLP), or a combination thereof. In some embodiments, the at least one cofactor is Flavin Adenine Dinucleotide (FAD). In some embodiments, the at least one cofactor is Pyridoxal 5′-phosphate (PLP). In some embodiments, the at least one cofactor is FAD and PLP.

In some embodiments, the at least one cofactor in the culture is in a concentration range of about 0.1 μM to about 1.5 μM. In some embodiments, the at least one cofactor in the culture is in a concentration range of about 0.25 μM to about 1 μM. In some embodiments, the cofactor is FAD at a concentration of about 0.5 μM. In some embodiments, the cofactor is FAD at a concentration of about 0.25 μM.

In some embodiments, the at least one cofactor in the culture is in concentration a range of about 1 μM to about 10 μM. In some embodiments, the at least one cofactor in the culture is in a concentration range of about 5 μM to about 10 μM. In some embodiments, the cofactor is PLP at a concentration of about 8 μM. In some embodiments, the cofactor is PLP at a concentration of about 4 μM.

In some embodiments, the contacting of the culture including human induced pluripotent stem cells (hiPSCs) with at least one cofactor is conducted when the hiPSCs are about 70-85% confluent. In some embodiments, the contacting of the culture including human induced pluripotent stem cells (hiPSCs) with at least one cofactor is conducted when the hiPSCs are about 70% confluent. In some embodiments, the contacting of the culture including human induced pluripotent stem cells (hiPSCs) with at least one cofactor is conducted when the hiPSCs are about 75% confluent. In some embodiments, the contacting of the culture including human induced pluripotent stem cells (hiPSCs) with at least one cofactor is conducted when the hiPSCs are about 80% confluent. In some embodiments, the contacting of the culture including human induced pluripotent stem cells (hiPSCs) with at least one cofactor is conducted when the hiPSCs are about 85% confluent.

In some embodiments, contacting the hiPSCs culture with FAD or PLP increases expression of PDX1. In some embodiments, contacting the hiPSCs culture with FAD or PLP increases expression of NKX6. In some embodiments, contacting the hiPSCs culture with FAD or PLP increases expression of NGN3.

In some embodiments, the concentration of the cofactor in the hiPSCs culture has to be in a range such that it is not too high and causes expression of PTF1a which is indicative of potential loss of endocrine lineage and exocrine differentiation.

In some embodiments, the hiPSCs culture is contacted with at least one cofactor during the pancreatic progenitor stage (S4) of differentiation of the hiPSCs. In such embodiments, the identification of expression of PDX1 and/or NKX6 and/or NGN3 is an indicator that the pancreatic progenitor stage (S4) cells will commit to differentiating into endocrine pancreatic β-cells. In some embodiments, the concentration of the cofactor used in the culture affects outcome as described in the Examples and Figures. For example, at certain concentrations of FAD and/or PLP, PTF1a may be expressed and indicates potential loss of endocrine lineage.

In some embodiments, the at least one cofactor is contacted with the hiPSCs culture for a period of at least 4 days. In some embodiments, the at least one cofactor is contacted with the hiPSCs culture for a period of about 1 day to about 7 days. In some embodiments, the at least one cofactor is contacted with the hiPSCs culture for a period of about 1 day. In some embodiments, the at least one cofactor is contacted with the hiPSCs culture for a period of about 2 days. In some embodiments, the at least one cofactor is contacted with the hiPSCs culture for a period of about 3 days. In some embodiments, the pancreatic progenitor cells are treated with the at least one cofactor for a period of about 4 days. In some embodiments, the at least one cofactor is contacted with the hiPSCs culture for a period of about 5 days. In some embodiments, the at least one cofactor is contacted with the hiPSCs culture for a period of about 6 days. In some embodiments, the at least one cofactor is contacted with the hiPSCs culture for a period of about 7 days.

### Compositions

Provided herein is a composition comprising endocrine pancreatic β-cells and at least one cofactor. Provided herein is a composition comprising hiPSCs, endocrine pancreatic β-cells, and at least one cofactor. Provided herein is a composition comprising hiPSCs in S4 stage of differentiation and at least one cofactor. In some embodiments, the at least one cofactor is FAD. In some embodiments, the at least one cofactor is PLP. In some embodiments, the at least one cofactor is a combination of FAD and PLP. In some embodiments, the composition is suitable for implanting/transplanting in a subject (e.g., an individual suffering from type 1 diabetes).

Also provided herein is a therapeutic product prepared according to the hiPSC culture methods described herein.

For therapeutic uses, the compositions may be supplied as part of a sterile, pharmaceutical composition that includes a pharmaceutically acceptable carrier. This composition can be in any suitable form (depending upon the desired method of administering it to a patient). The pharmaceutical composition can be administered to a patient by a variety of routes. The most suitable route for administration will depend on the particular a nature and severity of the disease and the physical condition of the subject.

Compositions can be conveniently presented in unit dosage forms containing a predetermined amount of β-cells described herein per dose. The quantity of β-cells included in a unit dose will depend on the disease being treated, the judgement of the physician, as well as other factors known in the art.

### Kits

Provided is a kit for use in producing endocrine pancreatic β-cells from a culture of human induced pluripotent stem cells (hiPSCs) comprising a composition comprising at least one cofactor.

In some embodiments, the at least one cofactor is FAD, PLP, or a combination thereof. In some embodiments, the at least one cofactor is FAD. In some embodiments, the at least one cofactor is PLP.

Such kits include one or more cofactors and instructions for use, e.g., instructions for administering a prophylactically or therapeutically effective amount of the cofactor. The cofactor may be in a vial or a pre-filled syringe. The kits may optionally further comprise means for administering the cofactor (e.g., an injection device, such as a pre-filled syringe), or means for measuring the outcome (e.g., measure blood sugar level).

In certain embodiments the individual components of the composition may be provided in one container, e.g., a vial or a pre-filled syringe. Alternatively, it may be desirable to provide the components of the pharmaceutical formulation separately in two or more containers, e.g., one container for the cofactor, and at least another container for a carrier compound. The kit may be packaged in a number of different configurations such as one or more containers in a single box. The different components can be combined, e.g., according to instructions provided with the kit. The kit can also include a delivery device.

### Treatment

Provided is a method for treating diabetes in a subject comprising implanting a therapeutically effective amount of endocrine pancreatic β-cells, prepared according to the methods described, in the subject in need thereof.

Provided is a method for regenerating β-cells in a subject, the method comprising implanting a therapeutically effective amount of endocrine pancreatic β-cells, prepared according to the methods described, in the subject in need thereof.

Provided is a method for treating pancreatic cancer in a subject, the method comprising implanting a therapeutically effective amount of endocrine pancreatic β-cells, prepared according to the methods described, in the subject in need thereof.

Accordingly, as described above and in the examples section, cofactors such as FAD and PLP may increase efficiency of β-cell regeneration by affecting the developmental pathways via SOX9 to increase the differentiation of pancreatic progenitor populations to the desired endocrine β-cells. The cofactors may act via promotion of SOX9 dimerization facilitating SOX9 binding to its genomic location. FAD and PLP, which are present in the human pancreas, could be sensors of the microenvironment and promote pancreatic regeneration when required.

### Examples

### Production of SOX9

Construct design and cloning: Codon-optimized, synthetic genes encoding full length SOX9 are acquired from GenScript, USA. A pair of oligonucleotides representing forward (5′-CAG TCA TAT GAA TCT GCT GGA TCC G-3′; SEQ ID NO:1) and reverse (5′-CAT ACT CGA GTC ATT TAT AGT CCG GAT G-3′; SEQ ID NO:2) sequences along with Ndel and Xhol restriction sites are synthesized (Integrated DNA Technologies) to amplify the SOX9 gene fragment (construct containing HMG and N-terminal domain, 1-173 amino acids) from full length gene using a standard polymerase chain reaction (PCR) protocol. The amplified PCR product is purified by agarose gel electrophoresis and subsequently digested with restriction enzymes Ndel and Xhol (New England Biolabs). The digested PCR product is ligated into pET-28a vector (Novagen), which is digested with the same set of restriction enzymes. The integration of construct with the vector is verified by DNA sequencing. Similarly, SOX9 HMG domain and other constructs are subcloned into pET-28a vector.

### Site Directed Mutagenesis

All the point mutations (A76E, H104A, K122A, K141A, H165A, K166A and K167A) in sox9 construct (1-173) are introduced by site-directed mutagenesis using an overlap extension PCR method. The QuickChange II XL Site-Directed Mutagenesis Kit (Agilent Technologies) is used and suitable oligonucleotide pairs purchased from Integrated DNA Technologies. DNA sequencing using vector specific primers in both the forward and reverse directions confirm the mutation at desired position.

### Protein Expression and Purification

For the expression of SOX9 (1-173) construct, a single colony of E. coli BL21(DE3)-RIL strain carrying the desired construct is inoculated in 100 mL of Luria-Bertani medium supplemented with 50 μg·mL−1 kanamycin and 50 μg·mL−1 chloramphenicol, and grown overnight at 37° C. in a rotary shaking incubator. An aliquot (1%) of the overnight-grown seed culture is inoculated in fresh Luria-Bertani medium supplemented with the same antibiotics and allowed to grow further until the absorbance (A) at 600 nm reaches a value of 0.6-0.8. The cultures are then down-tempered to 18° C. for 1 h before induction with 0.2 mM IPTG (isopropyl P-D-1-thiogalactopyranoside) for 16 h at 18° C. The cells are harvested by centrifuging the culture at 10,000 rpm for 15 min at 4° C. The cell pellet is re-suspended in lysis buffer (50 mM Tris-HCl, pH 8.0, 500 mM NaCl, 2 mM beta-mercaptoethanol (P-ME), and 20 mM Imadazole) along with a mixture of protease inhibitors and DNase I. The re-suspended cells are disrupted by sonication (30-s pulse on/off with 40% amplitude) (Sonics). The soluble and insoluble cell fractions are separated by centrifuging the cell lysate at 18,000 rpm for 60 min at 4° C. The supernatant is loaded onto a nickel-nitrilotriacetic acid (Ni-NTA) column that is pre-equilibrated with equilibration buffer (50 mM Tris-HCl, pH 8.0, 500 mM NaCl, and 20 mM Imadazole). The column is washed with 25 column volumes of wash buffer (50 mM Tris-HCl, pH 8.0, 500 mM NaCl, 2 mM P-ME, and 40 mM imidazole) to get rid of any nonspecifically bound proteins. Finally, the protein is eluted with elution buffer (50 mM Tris-HCl, pH 8.0, 150 mM NaCl, 2 P-ME, and 300 mM imidazole) and subsequently dialyzed overnight against 25 mM Tris-HCl, pH 8.0, 150 mM NaCl, 2 mM P-ME in cold room. The dialyzed protein is further purified by gel filtration chromatography using a Superdex 200 PG 16/30 column (GE Healthcare) equilibrated with 25 mM Tris-HCl, pH 8.0, 150 mM NaCl, 1 mM DTT. The purified protein is concentrated using an Amicon concentrator with a 10-kDa-molecular mass cutoff membrane and concentration is measured by UV 280 nm using calculated extinction coefficients. The mutant proteins A76E, H104A, K122A, K141A, H165A, K166A and K167A are expressed and purified similarly to wild type. The purity of the protein at each stage is checked by 4-20% SDS-PAGE.

### UV-Visible Spectroscopy

Purified SOX9 proteins of 2-4 mg/ml concentration are used for reading the absorbance of bind cofactors. The UV-Vis spectra are obtained on a UV-Vis spectrophotometer BMS (Biotechnology Medical Science) UV-1602, scanning from 300 nm to 800 nm. The baseline is corrected with the buffer used to prepare the protein samples.

### Sequence Alignment

Sequences for SOX9 (mouse, human) and several SOX members are obtained and aligned using Clustal Omega hosted by the EBI (ebi.ac.uk/Tools/msa/clustalo).

### Mass Spectrometry (MS)

MS data are analyzed and annotated with Genedata Expressionist software (v.13.0.1) using the Peptide Mapping Tool. The raw MS data are processed using two Genedata modules: Refiner MS for data pre-processing, and Analyst for data post-processing and statistical analyses. Briefly, after noise reduction, LC-MS1 base peaks are detected and their properties calculated (m/z and RT boundaries, m/z and RT center values, intensity). Chromatograms are further aligned based on the RT spectra using a 2 min range. Individual peaks are grouped into clusters and MS/MS data associated to these clusters are annotated with the Peptide Mapping Tool using the FASTA sequence of sp|Q04887|SOX9_MOUSE Transcription factor SOX-9. The search is performed with a peptide tolerance of 0.1 Da, a MS/MS tolerance of 0.01 Da, two allowed missed cleavages, and only annotations with a minimum score of 10 are considered. As variable modifications, the following parameters are applied: carbamidomethyl (C) maximum: 2 per sequence allowed: anywhere, FAD (C), FAD (H), FAD (Y) maximum: 2 per sequence allowed: anywhere, FMN (S), FMN (T) maximum: 2 per sequence allowed: anywhere, pyridoxal phosphate (K) maximum: 2 per sequence allowed: anywhere, and pyridoxal phosphate H2 (K) maximum: 2 per sequence allowed: anywhere.

### Cellular Assay

Human induced pluripotent stem cells (hiPSCs) are cultured and maintained using mTeSR Plus medium (Stem Cell Technologies, Canada), then plated on 1:50 Matrigel-coated dishes (Corning, USA). Upon reaching 70-80% confluency, cells are differentiated into pancreatic progenitors using a protocol described by Memon et al. (Memon et al. Stem Cell Research and Therapy, 9, Article 15, 2018). Cells are treated with different concentrations of FAD (0.25 μM, 0.5 μM, and 1 μM) (Sigma-Aldrich, Germany) during the four days of pancreatic progenitor stage of in vitro differentiation. The control cells are treated with water.

### Immunostaining and Western Blotting

After the treatment period, cells are fixed and immunostaining is performed using previously reported protocols by Memon et al. Cells are also collected for total protein extraction using Radioimmunoprecipitation Assay (RIPA) lysis buffer and are normalized to 20 ng/μL before SDS-PAGE separation. Nitrocellulose membranes are blocked using 15% skimmed milk in 0.5% tris-buffered saline (TBST). Membranes are then incubated with primary antibodies overnight at 4° C. Membranes are washed with 0.5% TBST and secondary antibodies are added for 1 hour at room temperature. Membranes are developed using SuperSignal West Pico Chemiluminescent substrate (Pierce, Loughborough, UK) and are then visualized using iBright™ CL 1000 Imaging System (Invitrogen).

### Results

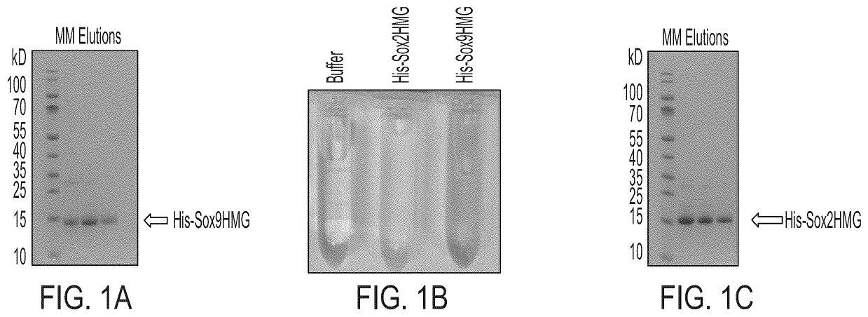

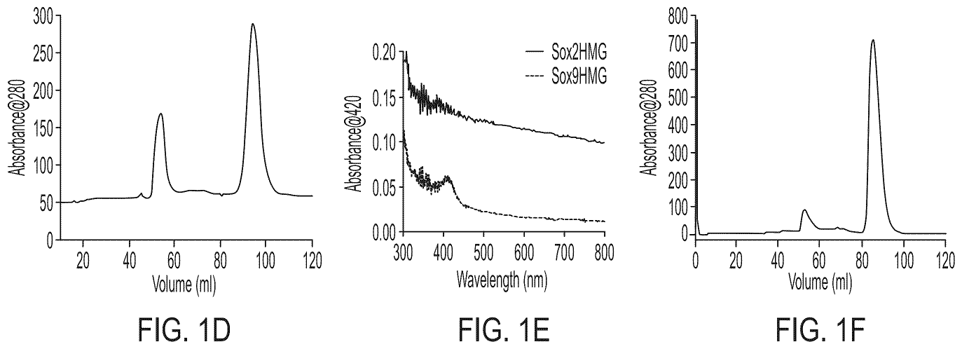

SOX9 HMG domain is produced using bacterial expression (FIG. 1A) and the purified protein exhibits a yellowish-brown color previously not observed with SOX2 HMG domain protein obtained in a similar manner (FIG. 1C). Yields from expression are similar for all forms of HMG domains. All the proteins also have high purity (FIG. 1D and FIG. 1F) and the molecular sizes of these highly conserved HMG domains (SOX9 and SOX2) are similar (˜15 kD).

Further analysis (FIG. 1B) employing absorbance spectroscopy shows a peak at approximately 420 nm characteristic of an FAD-bound protein for SOX9 HMG domain (FIG. 1E). This peak is pronounced and significant relative to the spectrum. The profile for SOX2 HMG domain shows no obvious peak.

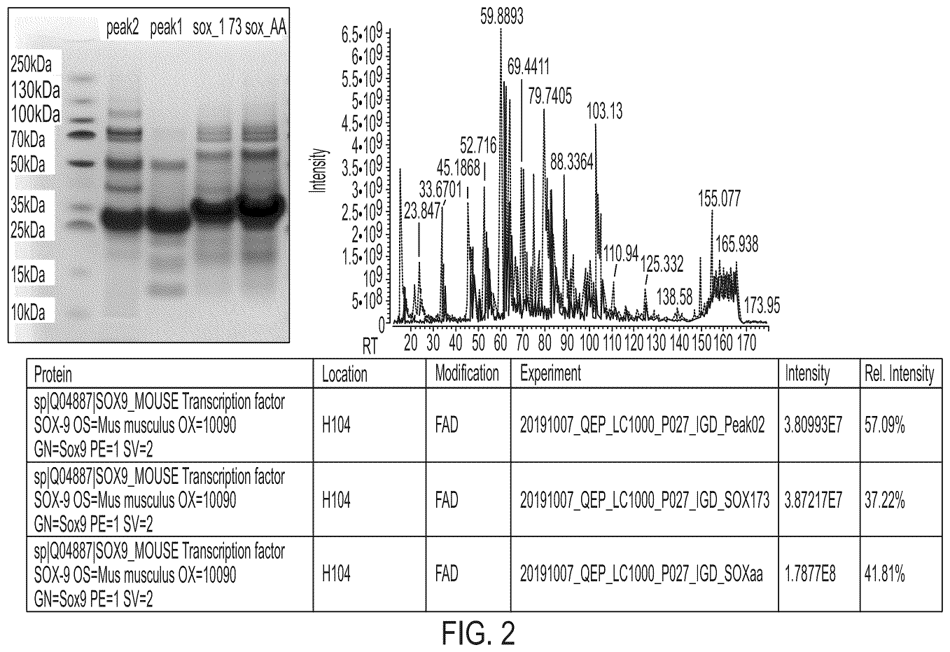

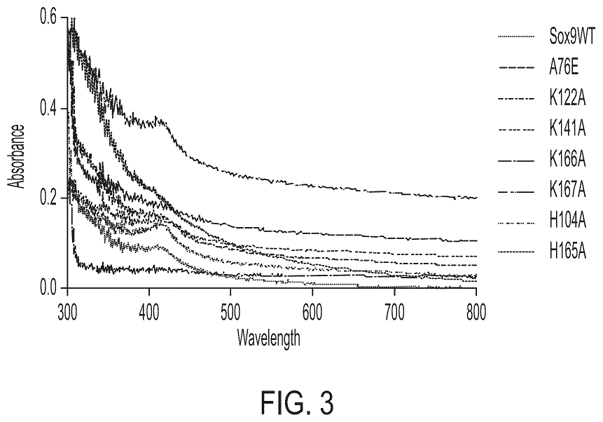

The purified SOX9 HMG domain is subjected to 1D-PAGE in-gel digest and LC-MS/MS (FIG. 2) to identify the putative cofactor giving rise to the yellow color of the protein. Several peaks are detected suggestive of FAD, FMN and PLP binding due to the changed intensity profile for specific peptide fragments. Six peptide sequences corresponding to either FAD or PLP binding with high probability are identified with 6 specific residues (K122, K141, K166, K167, H104, H165) potentially responsible for binding the cofactor (FIG. 3). These specific residues are chosen because they are all within the HMG domain and all have both mass and MS/MS confirmation. Sequence comparisons are made between SOX9 and several other SOX family members. The homology/identity of the HMG domains is high (>90%). H104 and K166 are conserved only within SOXE family members (SOX8,9,10) and not observed with other SOX family members. The A76 residue, associated with dimerization, is also found only in SOXE family members.

Site-directed mutants of the six residues (individually converted to alanine) as well as the previously reported dimerization mutant (A76E) of SOX9 HMG domain are purified and characterized by absorbance spectroscopy. All mutants show an absorbance peak similar to wild type SOX9 HMG domain except for H104A, H165A and A76E where the peak is completely absent (FIG. 3).

Structural analysis of available SOX9 HMG domain structures (complexed with cognate DNA) shows that both the mouse (4S2Q) and human (4EUW) SOX9 structures are highly similar (rmsd 1A). Inspection of the dimerization domain and residues H104 and H165 shows the residues can putatively participate in a dimerization as they all lie on the same side of SOX9.

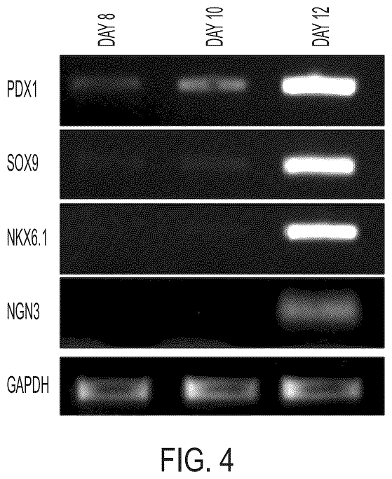

FIG. 4 shows gene expression of differentiated cells during early stages of pancreatic differentiation (days 8, 10 and 12). High expression of PDX1, SOX9 and NKX6.1 is observed at day 12 of differentiation. GAPDH is used as housekeeping gene.

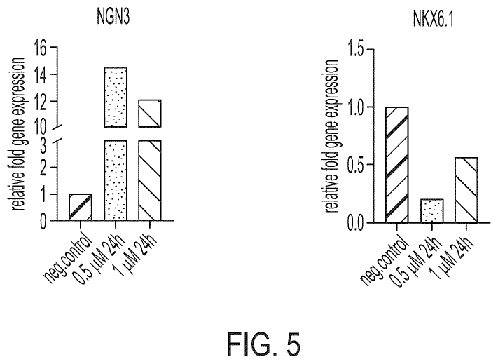

FIG. 5 shows relative changes in NGN3 and NKX6.1. Addition of FAD to early stage differentiation appears to increase pancreatic developmental factors.

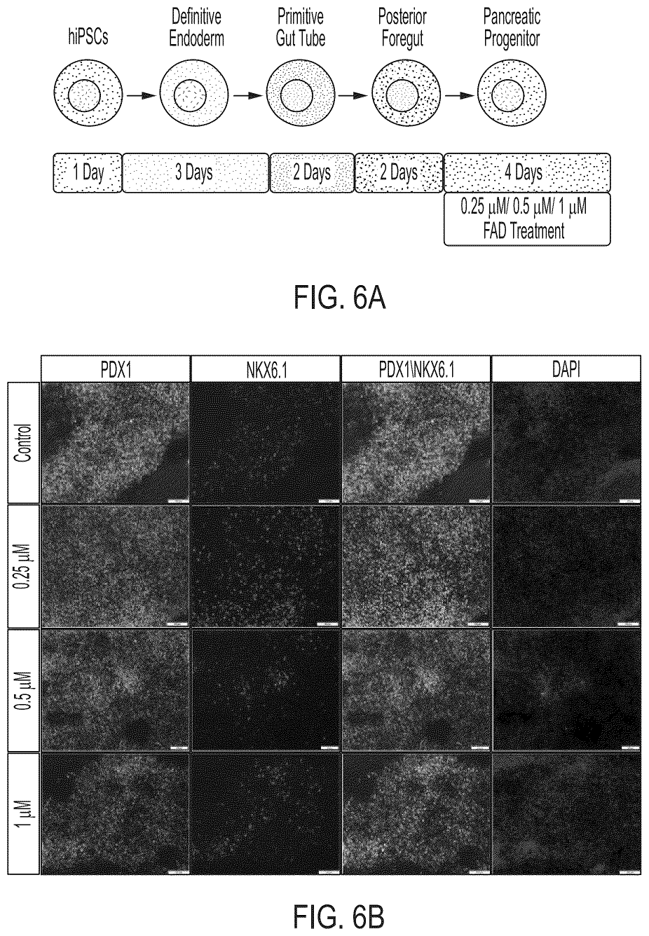

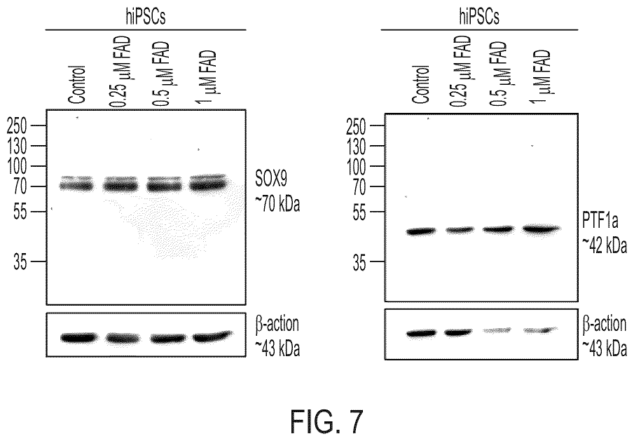

hiPSC-derived pancreatic progenitors are generated in vitro and are treated with different FAD concentrations during the four days of pancreatic progenitor stage of pancreatic differentiation. The efficiency of pancreatic progenitor differentiation is determined by examining the expression of PDX1 and NKX6.1. Immunostaining shows that most of the pancreatic progenitor cells co-expressed PDX1 and NKX6.1 (FIG. 6), indicating a high differentiation efficiency. Western blotting shows increased levels of the transcription factors, SOX9 and PTF1a, upon FAD treatment (FIG. 7). SOX9 increases gradually with increased FAD concentrations, while PTF1a, which is a key exocrine marker, is increased in 0.5 μM and 1 μM FAD-treated pancreatic progenitors.

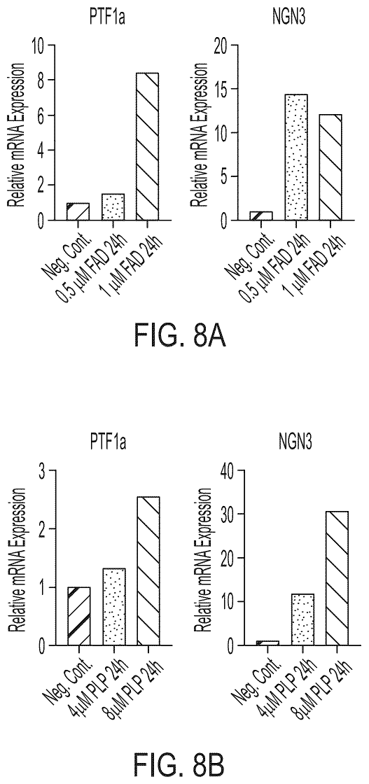

FIG. 8A shows expression of PTF1a at about 0.5 μM concentration of FAD along with NGN3. PTF1a expression may correlate with loss of endocrine lineage and is less desirable. Accordingly a concentration of about 0.5 μM or lower, such as 0.5 μM, is preferred when FAD is contacted with hiPSCs using the methods described. FIG. 8B shows appearance of PTF1a at about 4 μM-8 μM of PLP, but also a high expression of NGN3. Accordingly a concentration of about 8 μM or lower, is preferred when PLP is contacted with hiPSCs using the methods described.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

We claim:

1. A method for producing endocrine pancreatic β-cells, the method comprising
contacting a culture including human induced pluripotent stem cells (hiPSCs) with at least one cofactor;
differentiating at least a portion of the hiPSCs to endocrine pancreatic β-cells; and
measuring expression of a biomarker selected from PDX1, NKX6, NGN3, or a combination thereof to identify the endocrine pancreatic β-cells, wherein expression of said biomarkers indicates differentiation to endocrine pancreatic β-cells.

2. The method of claim 1, wherein the hiPSCs are contacted with at least one cofactor during the pancreatic progenitor stage (S4) of differentiation of the hiPSCs.

3. The method of claim 2, wherein the at least one cofactor is contacted with the hiPSCs for a period of at least 4 days.

4. The method of claim 1, wherein the at least one cofactor is Flavin Adenine Dinucleotide (FAD), Pyridoxal 5′-phosphate (PLP), or a combination thereof.

5. The method of claim 1, wherein the at least one cofactor is FAD.

6. The method of claim, wherein the at least one cofactor is PLP.

7. The method of claim 1, wherein the at least one cofactor is in a concentration range of about 0.1 μM to about 1.5 μM.

8. The method of claim 1, wherein the at least one cofactor is in a concentration range of about 0.25 μM to about 1 μM.

9. The method of claim 1, wherein the at least one cofactor is FAD at a concentration of about 0.5 μM.

10. The method of claim 1, wherein the at least one cofactor is in a concentration range of about 1 μM to about 10 μM.

11. The method of claim 1, wherein the at least one cofactor is in a concentration range of about 5 μM to about 10 μM.

12. The method of claim 1, wherein the at least one cofactor is PLP at a concentration of about 8 μM.

13. The method of claim 1, wherein the contacting of the culture including human induced pluripotent stem cells (hiPSCs) with at least one cofactor is conducted when the hiPSCs are about 70% confluent.

14. A kit for use in producing endocrine pancreatic β-cells from a culture of human induced pluripotent stem cells (hiPSCs) comprising a composition comprising at least one cofactor.

15. A method for treating diabetes in a subject comprising implanting a therapeutically effective amount of endocrine pancreatic β-cells, prepared according to the method of claim 1, in the subject in need thereof.

16. A method for regenerating β-cells in a subject, the method comprising implanting a therapeutically effective amount of endocrine pancreatic β-cells, prepared according to the method of claim 1, in the subject in need thereof.

17. A method for treating pancreatic cancer in a subject, the method comprising implanting a therapeutically effective amount of endocrine pancreatic β-cells, prepared according to the method of claim 1, in the subject in need thereof.

18. A composition comprising endocrine pancreatic β-cells and at least one cofactor.

19. The composition of claim 18, wherein the at least one cofactor is FAD, PLP, or a combination thereof.

20. A therapeutic product prepared according to the method of claim 1.
